(19) United States
(12) Patent Application Publication
(10) Pub. No.: US 2019/0117106 A1
(43) Pub. Date: Apr. 25, 2019

(54) PROTOCOL AND SIGNATURES FOR THE MULTIMODAL PHYSIOLOGICAL STIMULATION AND ASSESSMENT OF TRAUMATIC BRAIN INJURY

(71) Applicant: Cerora, Inc., Bethehe,m, PA (US)

(72) Inventors: Adam Simon, Yardley, PA (US); David M. Devilbiss, Madison, WI (US)

(21) Appl. No.: 16/094,551

(22) PCT Filed: Apr. 18, 2017
(86) PCT No.: PCT/US17/28147

§ 371 Date: Oct. 18, 2018

Related U.S. Application Data
(60) Provisional application No. 62/324,269, filed on Apr. 18, 2016.

(51) Int. Cl. A61B 5/0484; A61B 5/00

(52) U.S. Cl. CPC A61B 5/0484; A61B 5/7257; A61B 5/7207; A61B 5/4064

(57) ABSTRACT

A system and method for assessing brain function is disclosed that comprises electronically recording biologic information of a user with one or more electronics modules as the user progresses through a series of cognitive, sensory, activation, and/or stimulation tasks. The method includes extracting one or more data features from the record biologic information to obtain extracted data features. The method includes analyzing the extracted data features for each task so to develop a predictive outcome assessment of one or more brain conditions of the user, wherein predictive outcome assessment is at least one of a) an injury determination, b) a brain injury index, or c) a brain health assessment. Medical therapy is provided to the user in accordance with the predictive outcome assessment.

### CROSS-REFERENCE TO RELATED APPLICATIONS

The present application claims priority to U.S. Provisional Patent Application No. 62/324,269, filed Apr. 18, 2016. The contents of that application are hereby incorporated by reference.

### TECHNICAL FIELD

The present disclosure relates to diagnosis and analysis of brain health through the use of activated tasks and stimuli in a system to dynamically assess one's brain state and function.

### BACKGROUND

Normal functioning of the brain and central nervous system is critical to a healthy, enjoyable and productive life. Disorders of the brain and central nervous system are among the most dreaded of diseases. Many neurological disorders such as stroke, Alzheimer's disease, and Parkinson's disease are insidious and progressive, becoming more common with increasing age. Others such as schizophrenia, depression, multiple sclerosis and epilepsy arise at younger age and can persist and progress throughout an individual's lifetime. Sudden catastrophic damage to the nervous system, such as brain trauma, infections and intoxications can also affect any individual of any age at any time.

Most nervous system dysfunction arises from complex interactions between an individual's genotype, environment and personal habits and thus often presents in highly personalized ways. However, despite the emerging importance of preventative health care, convenient means for objectively assessing the health of one's own nervous system have not been widely available. Therefore, new ways to monitor the health status of the brain and nervous system are needed for normal health surveillance, early diagnosis of dysfunction, tracking of disease progression and the discovery and optimization of treatments and new therapies.

Unlike cardiovascular and metabolic disorders, where personalized health monitoring biomarkers such as blood pressure, cholesterol, and blood glucose have long become household terms, no such convenient biomarkers of brain and nervous system health exist. Quantitative neurophysiological assessment approaches such as positron emission tomography (PET), functional magnetic resonance imaging (fMRI) and neuropsychiatric or cognition testing involve significant operator expertise, inpatient or clinic-based testing and significant time and expense. One potential technique that may be adapted to serve a broader role as a facile biomarker of nervous system function is a multi-modal assessment of the brain from a number of different forms of data, including electroencephalography (EEG), which measures the brain's ability to generate and transmit electrical signals. However, formal lab-based EEG approaches typically require significant operator training, cumbersome equipment, and are used primarily to test for epilepsy.

Alternate and innovative biomarker approaches are needed to provide quantitative measurements of personal brain health that could greatly improve the prevention, diagnosis and treatment of neurological and psychiatric disorders. Unique multimodal devices and tests that lead to biomarkers of Parkinson's disease, Alzheimer's disease, concussion and other neurological and neuropsychiatric conditions is a pressing need.

### SUMMARY

A system, device and method for assessing brain function is disclosed that comprises electronically recording biologic information of a user with one or more electronics modules as the user progresses through a series of cognitive, sensory, activation, and/or stimulation tasks. The method includes extracting one or more data features from the record biologic information to obtain extracted data features. The method includes analyzing the extracted data features for each task so to develop a predictive outcome assessment of one or more brain conditions of the user, wherein predictive outcome assessment is at least one of a) an injury determination, b) a brain injury index, or c) brain health assessment. Medical therapy is provided to the user in accordance with the predictive outcome assessment.

In an exemplary embodiment, the system includes one or more electronics modules configured to be mounted on the user. The electronics modules include an active brainwave sensor that collects at least one channel of an electroencephalography (EEG) brainwave data stream. A plurality of biological sensors are also provided that simultaneously record biological sensor data from the user. The plurality of biological sensors include a microphone that records human speech to capture verbal responses of the human subject during the series of tasks, and an image sensor that records eye movements, eye saccade and biometric identification information. A stimulation device is also provided that applies at least one of a visual stimulant, an auditory stimulant, a gastronomic stimulant, an olfactory stimulant, and/or a motion stimulant to the user. During use, the plurality of biological sensors simultaneously measure the user's response to stimulants applied by the stimulation device in accordance with at least one task that causes statistically different results between brain injured subjects and brain non-injured subjects for recordation by the electronics module.

In the exemplary embodiments, the tasks shown to cause statistically different results between brain injured subject and brain non-injured subjects include a binaural 12 Hz beat task. During these tasks, the electronics module measures at least one of relative power in a 38-40 Hz range during a binaural 12 Hz beat task, relative power in a 30-45 Hz range during a binaural 12 Hz beat task, and a relative theta power during a binaural 12 Hz beat task. The statistically different tasks also include at least an eyes closed task where the electronics module measures relative 4-6 Hz power or relative theta-lower power during the eyes closed task; a Standardized Assessment of Concussion (SAC)-delayed recall task where the electronics module measures artifact during the SAC-delayed recall task; a Standardized Assessment of Concussion (SAC)-concentration task where the electronics module measures relative 54-56 Hz power during the SAC-concentration task; a Balance Error Scoring System (BESS) firm surface task where the electronics module measures at least one of absolute 46-48 Hz power during the BESS firm surface task and absolute 48-50 Hz power during the BESS firm surface task; a binaural 6 Hz beat task where the electronics module measures a 6 Hz binaural beat primary driving frequency and a first harmonic; and/or a binaural 12 Hz beat task where the electronics module measures a 12 Hz binaural beat primary driving frequency and a first harmonic and/or a second harmonic.

### BRIEF DESCRIPTION OF THE DRAWINGS

Embodiments of the present disclosure can be better understood with reference to the following drawings.

FIG. 1A-1B are schematic diagrams illustrating the sixteen (16) tasks which comprise the Lehigh protocol of concussion assessment, including a block diagram (FIG. 1B) which does not show the initial “Welcome” task or the “pause” task between the BESS-foam surface and the delayed recall task.

FIG. 2 is a tabular representation of the interim analysis demographics including the number of baseline, controls, as well as and concussed/traumatic brain injury subjects.

FIG. 3A graphical representation of the number of baseline subjects who had a given symptom in the Graded Symptom Checklist (N=230 baseline subjects).

FIG. 3B is a scatter and box-whisker plot of the number of symptoms gathered in the GSC.

FIG. 3C is a scatter and box-whisker plot of the SAC total score (out of a possible 30 maximum).

FIG. 3D is a scatter and box-whisker plot of the BESS total errors after 6 positions for 20 seconds each.

FIG. 3E is a scatter and box-whisker plot of the K-D test (2×3 test) total number of errors.

FIG. 3F is a scatter and box-whisker plot of the K-D test (2×3 test) total time in seconds.

FIG. 4A is a scatter and box-whisker plot for each of the 5 primary EEG frequency bands for each of the 16 tasks described in the Lehigh Protocol (FIG. 1).

FIG. 4B is a graphical representation of power spectra from seven (7) subjects 7 subjects during EC shows prominent alpha rhythm peak around 10 Hz.

FIG. 4C is a graphical representation of the mean of the EC/EO ratio for all N=230 baseline subjects.

FIG. 4D is a graphical representation of the 6 Hz and 12 Hz binaural beat stimulation task power spectra measured at baseline showing primary frequency and first harmonic elevations.

FIG. 5 is an ensemble of Krippendorff alphas, a generalization of Pearson's Intraclass Correlation Coefficient, designed to account for missing data. In each case, the distribution of N=1000 bootstrapped trials is shown with the mean value identified above the distribution as an “effective” ICC.

FIG. 6A is a graphical presentation of the two group comparison of the GSC total severity score between concussed and control subjects.

FIG. 6B is a graphical presentation of the two group comparison of the SAC total score between concussed and control subjects.

FIG. 6C is a graphical presentation of the two group comparison of the BESS total score between concussed and control subjects.

FIG. 6D is a graphical presentation of the two group comparison of the K-D (2×3) Test total time (seconds) between concussed and control subjects.

FIG. 6E is a tabular representation of the two group comparisons of FIG. 5A thru FIG. 5D showing the median values of each parameter in control and concussed groups as well as statistical significance of the difference as determined by the Wilcoxon signed-rank test false positive rate (FPR) p-value.

FIG. 7A is a graphical presentation of the two group comparison of the EEG relative theta band power (rTheta) during the 12 Hz Binaural Beat auditory stimulation task.

FIG. 7B is a graphical presentation of the two group comparison of the EEG relative theta band power (rTheta) during the 6 Hz Binaural Beat auditory stimulation task.

FIG. 7C is a graphical presentation of the two group comparison of the EEG relative theta band power (rTheta) during the K-D (2×3) test task.

FIG. 7D is a graphical presentation of the two group comparison of the EEG relative alpha band power (rAlpha) during the Eyes Closed (EC) task.

FIG. 7E is a tabular representation of the two group comparisons of FIG. 6A thru FIG. 6D showing the median values of each parameter in control and concussed groups as well as statistical significance of the difference as determined by the Wilcoxon signed-rank test false positive rate (FPR) p-value.

### DETAILED DESCRIPTION OF ILLUSTRATIVE EMBODIMENTS

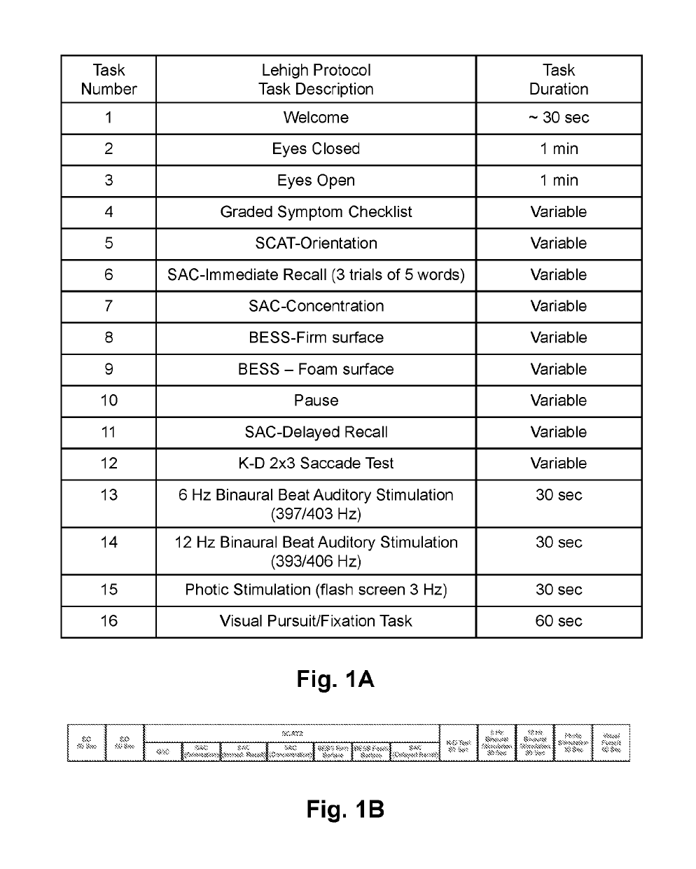

The present disclosure will be described in detail below with reference to FIGS. 1-7. Those skilled in the art will appreciate that the description given herein with respect to those figures is for exemplary purposes only and is not intended in any way to limit the scope of the present disclosure. All questions regarding the scope of the present disclosure may be resolved by referring to the appended claims.

### Definitions

By “electrode to the scalp” we mean to include, without limitation, those electrodes requiring gel, dry electrode sensors, contactless sensors and any other means of measuring the electrical potential or apparent electrical induced potential by electromagnetic means.

By “monitor the brain and nervous system” we mean to include, without limitation, surveillance of normal health and aging, the early detection and monitoring of brain dysfunction, monitoring of brain injury and recovery, monitoring disease onset, progression and response to therapy, for the discovery and optimization of treatment and drug therapies, including without limitation, monitoring investigational compounds and registered pharmaceutical agents, as well as the monitoring of illegal substances and their presence or influence on an individual while driving, playing sports, or engaged in other regulated behaviors.

A “medical therapy” as used herein is intended to encompass any form of therapy with potential medical effect, including, without limitation, any pharmaceutical agent or treatment, compounds, biologics, medical device therapy, exercise, biofeedback or combinations thereof, or changes or alterations to the next diagnostic procedures.

By “EEG data” we mean to include without limitation the raw time series, any spectral properties determined after Fourier transformation, any nonlinear properties after non-linear analysis, any wavelet properties, any summary biometric variables and any combinations thereof.

A “sensory and cognitive challenge” as used herein is intended to encompass any form of sensory stimuli (to the five senses), cognitive challenges (to the mind), and other challenges (such as a respiratory CO₂ challenge, virtual reality balance challenge, hammer to knee reflex challenge, etc.).

A “sensory and cognitive challenge state” as used herein is intended to encompass any state of the brain and nervous system during the exposure to the sensory and cognitive challenge.

An “electronic system” as used herein is intended to encompass, without limitation, hardware, software, firmware, analog circuits, DC-coupled or AC-coupled circuits, digital circuits, FPGA, ASICS, visual displays, audio transducers, temperature transducers, olfactory and odor generators, or any combination of the above.

By “spectral bands” we mean without limitation the generally accepted definitions in the standard literature conventions such that the bands of the PSD are often separated into the Delta band (f<4 Hz), the Theta band (4<f<7 Hz), the Alpha band (8<f<12 Hz), the Beta band (12<f<30 Hz), and the Gamma band (30<f<100 Hz). The exact boundaries of these bands are subject to some interpretation and are not considered hard and fast to all practitioners in the field.

By “calibrating” we mean the process of putting known inputs into the system and adjusting internal gain, offset or other adjustable parameters in order to bring the system to a quantitative state of reproducibility.

By “conducting quality control” we mean conducting assessments of the system with known input signals and verifying that the output of the system is as expected. Moreover, verifying the output to known input reference signals constitutes a form of quality control which assures that the system was in good working order either before or just after a block of data was collected on a human subject.

By “biomarker” we mean an objective measure of a biological or physiological function or process.

By “biomarker features or metrics” we mean a variable, biomarker, metric or feature which characterizes some aspect of the raw underlying time series data. These terms are equivalent for a biomarker as an objective measure and can be used interchangeably.

By “non-invasively” we mean lacking the need to penetrate the skin or tissue of a human subject

By “diagnosis” we mean any one of the multiple intended use of a diagnostic including to classify subjects in categorical groups, to aid in the diagnosis when used with other additional information, to screen at a high level where no a priori reason exists, to be used as a prognostic marker, to be used as a disease or injury progression marker, to be used as a treatment response marker, or even as a treatment monitoring endpoint.

By “electronics module” or “EM” or “reusable electronic module” or “REM” or “multi-functional biosensor” or “MFB” we mean an electronics module or device that can be used to record biological signals from the same subject or multiple subjects at different times. By the same terms, we also mean a disposable electronics module that can be used once and thrown away which may be part of the future as miniaturization becomes more common place and costs of production are reduced. The electronics module can have only one sensing function or a multitude (more than one), where the latter (more than one) is more common. All of these terms are equivalent and do not limit the scope of the present disclosure.

By “biosignals” or “bio signals” or “bio-signals” we mean any direct or indirect biological signal measurement data streams which either directly derives from the human subject under assessment or indirectly derives from the human subject. Non-limiting examples for illustration purposes include EEG brainwave data recorded either directly from the scalp or contactless from the scalp, core temperature, physical motion or balance derived from body worn accelerometers, gyrometers, and magnetic compasses, the acoustic sound from a microphone to capture the voice of the individual, the stream of camera images from a front facing camera, the heart rate, heart rate variability and arterial oxygen from a would pulse oximeter, the skin conductance measured along the skin, the cognitive task information recorded as keyboard strokes, mouse clicks or touch screen events. There are many other biosignals to be recorded as well.

By “Return to Play” we mean similar decisions such as return to duty, return to work, return to learn, return to drive, insurance coverage decision (return to coverage) or any other return to activity based decision that has a different context but is essentially the same question about a human subject trying to return to an earlier state to resume an “activity” that they participated in previously.

By “Lehigh Protocol” we mean the combination of tasks or subsets therein of those tasks listed in FIG. 1A and FIG. 1B used in a single scan session to assess the brain health and function of a subject or patient.

### A System of Multiple Transducers to Both Stimulate and Record Physiological and Brain Response

As described in earlier patent applications by the present inventors, the systems and methods of the present disclosure comprise multiple transducers to both stimulate and record the physiological response of the brain and the body in order to assess its health and function. Central to the system is the ability to directly record brainwave activity from an electrode placed non-invasively on or near the scalp. Moreover, additional information on brain health and function can be derived from transducers that measure position and motion, temperature, cardiovascular properties like heart rate, heart rate variability, and arterial oxygen, as well as cognitive information, speech, eye movement, and surface skin conductance to name a few non-limiting additional biological signal measurement data stream examples. It is often necessary to bring the system to the human subject, getting out of the hospital or doctor's office and enabling data collection in the home or sports field or combat theater, thus providing accessibility to the brain health and function assessment from a lightweight and portable form factor. Moreover, it would be advantageous to have a minimal cost associated with the system so that it can be used around the globe to help those in need of brain health and function assessments and to provide the appropriate medical treatment.

One embodiment is a system for capturing multiple streams of biological sensor data for assessing brain health of a user. The system includes an electronics module mounted on or near the user's head including an active brainwave sensor that collects at least one channel of EEG brainwave data. The system also includes a plurality of biological sensors that simultaneously record biological sensor data from the user using a plurality of biological sensors. The biological sensors includes a microphone that records human speech to capture verbal responses of the human subject during a battery of tasks to either cognitive challenges or auditory stimulations and an image or motion tracking sensor that records that records eye movements, eye saccade and other biometric identification information. The biological sensors can include a 3-axis accelerometer or 6-axis accelerometer/gyrometry combination that enables the measurement of both static and dynamic measures of postural stability. The system also includes a stimulation device that applies at least one of a visual stimulant, an auditory stimulant, a gastronomic stimulant, an olfactory stimulant, and/or a motion stimulant to the user. The plurality of biological sensors is configured to simultaneously measure the body's response to stimulants applied by said stimulation device for recordation by the electronic module. The system as used here includes systems, devices, and methods as disclosed in U.S. patent application Ser. No. 14/773,872, filed Sep. 9, 2015, the entire contents of which are incorporated herein by reference.

### Use of the Multimodal System to Create Multivariate Signatures of Disease or Injury

Using the system of the present disclosure, one can build extracted biometric tables after signal preprocessing and signal processing that include features extracted from multiple modes of biological signal data. As a non-limiting example, two groups of subjects, group A who experienced a concussion (mTBI) or mild traumatic brain injury, and group B who did not and serve as Controls (CTL), were recruited under the supervision of an Institutional Review Board. Participants from both groups A and B were scanned identically with an electronic REM module including a single electrode EEG. A 22 to 24 minute protocol was implemented including those tasks listed in FIG. 1. The stop watch times and errors for each card of the saccade test were recorded manually by the test administrator while the peripheral MCU (a laptop computer) presented the cards and recorded the acoustic and auditory responses of the individuals via the microphone. The data was blinded to participant for the purposes of artifact detection, signal processing and feature extraction. The extracted feature data table was then quality controlled and scrubbed to remove as many errors as possible.

Each of the key biomarkers such as the Graded Symptom Checklist (GSC) total severity and total number of symptoms was scored independently. The Standardized Assessment of Concussion (SAC) score was noted, the total Balance Error Scoring System (BESS) errors were noted, as was both the total saccade time as well as errors or other outcome measures for that given saccade test.

Athletes from a collegiate university were scanned according to the protocol shown in FIG. 1. The task battery was selected from amongst tasks published in the literature as well as novel tasks in accordance with aspects of the present disclosure. The computer system and the field clinician administered each task and the data was recorded by the software as well as on paper to cross validate computer stored findings. The data were then encrypted locally with SilverKey by AES-256 bit encryption and transported to the cloud for analysis.

Once in the cloud, each encrypted parcel was decrypted and analyzed. Each trace of data recorded went through both a preprocessing phase to remove artifacts as well as then a signal processing phase to extract features of the signal time series data. The most common analysis was spectral or Fast Fourier Transform (FFT) analysis although both discrete and continuous wavelet analysis was conducted as well (see: Ghorbanian P, Devilbiss D M, Hess T, Bernstein A, Simon, A J, Ashrafiuon H. Identification of resting and active state EEG features of Alzheimer's disease using discrete wavelet transform. Ann Biomed Eng. 2013 June; 41(6): 1243-57, as well as Ghorbanian P, Devilbiss D M, Hess T, Bernstein A, Simon, A J, Ashrafiuon H. Exploration of EEG features of Alzheimer's disease using continuous wavelet transform. Med Biol Eng Comput, published online 12 Apr. 2015.) Non-linear dynamical analysis was also conducted in some instances.

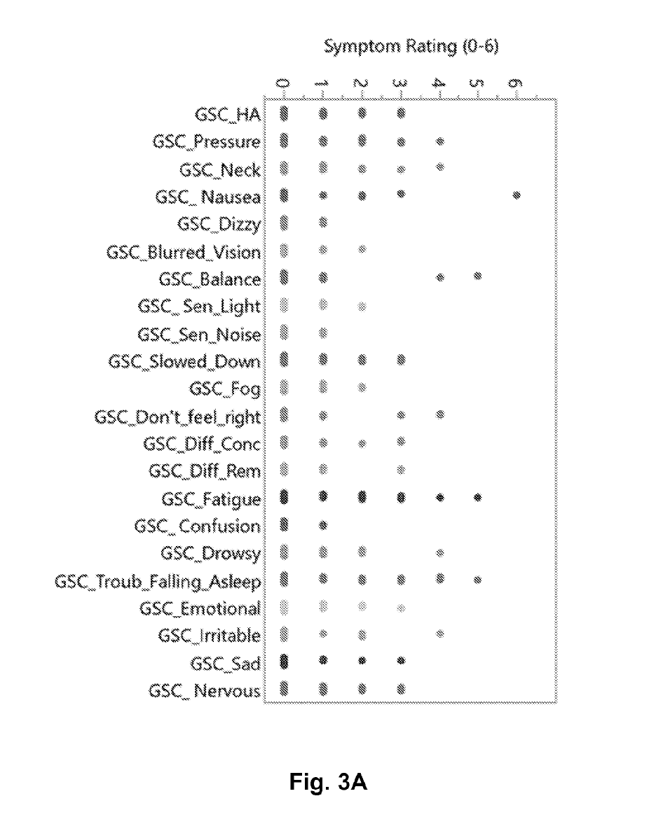

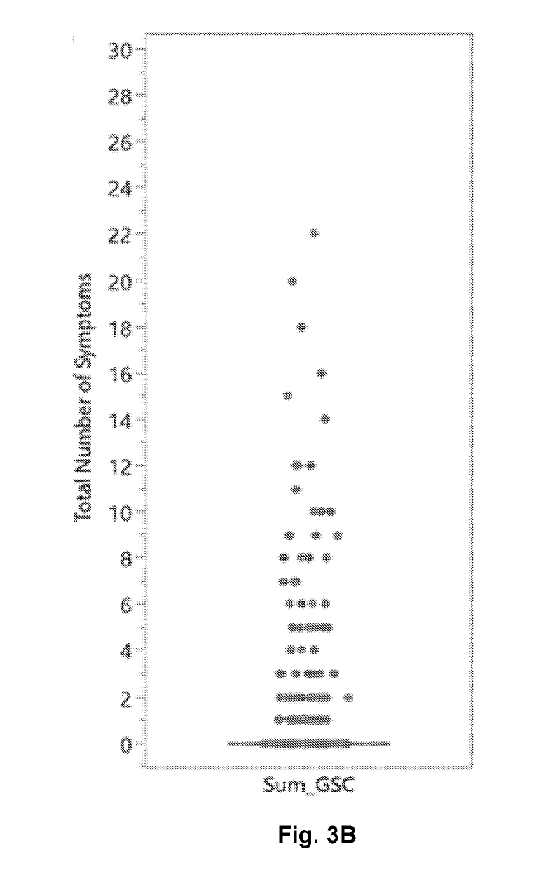

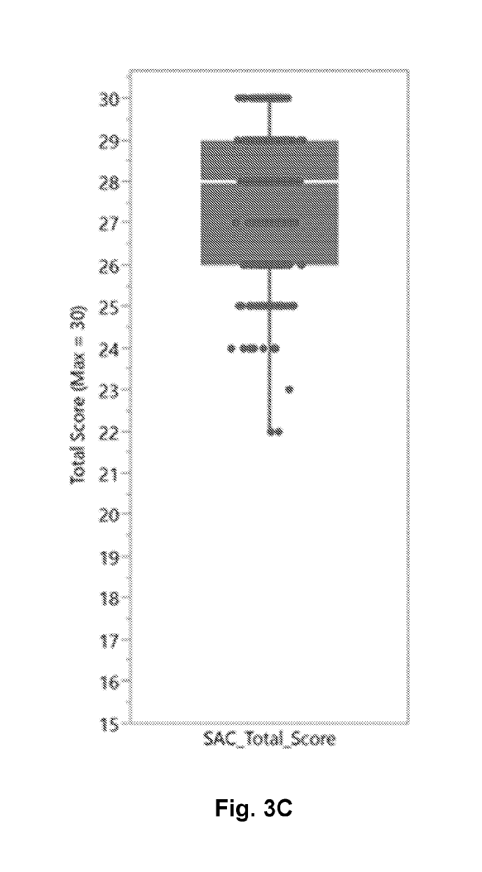

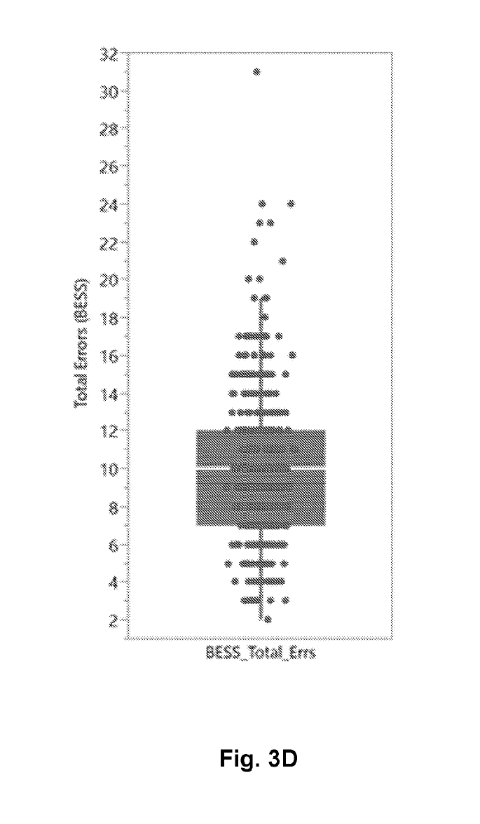

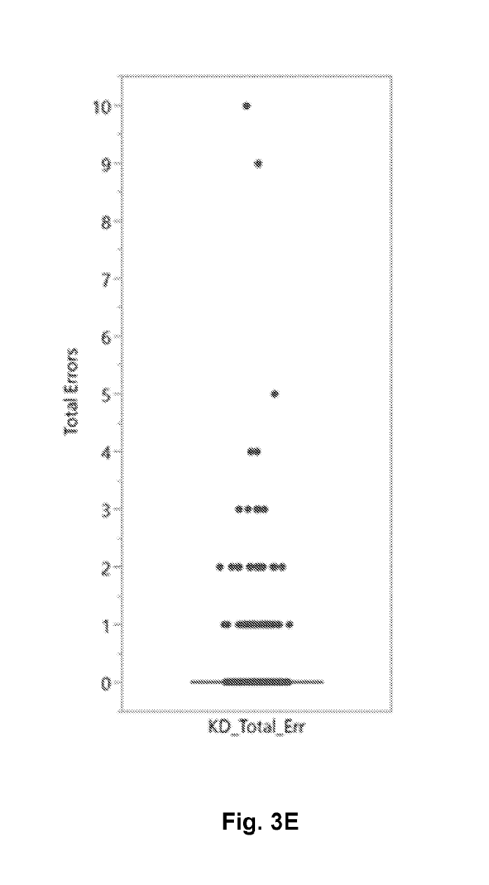

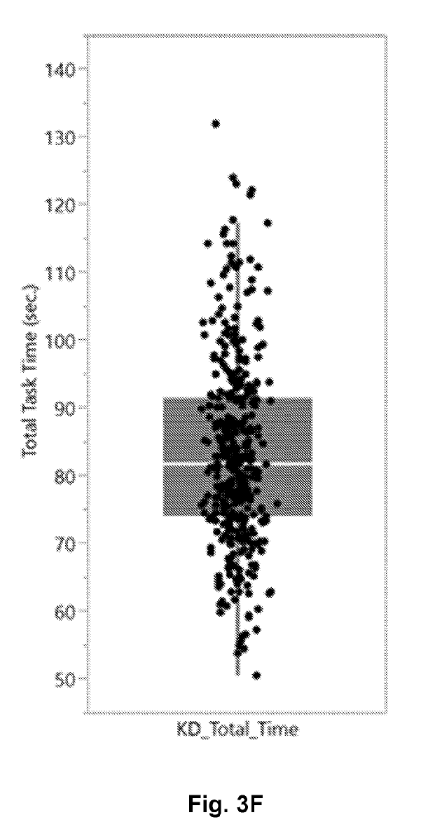

Feature data from published literature was evaluated first for baseline scans of athletes. FIG. 3A shows the symptoms reported and their severity for the 230 baseline subjects. FIG. 3B shows the number of symptoms rather than the severity in baseline subjects. In FIG. 3C, the Standard Assessment of Concussion is reported in the baseline subjects with a maximum possible score of 30 points. In FIG. 3D, the total number of Balance Error Scoring System (BESS) errors is reported. In FIG. 3E, the number of King-Devick test (or 2×3 Saccade test) is graphically shown for all baseline subjects. Lastly, the total time to read the best 3 card set is reported in a FIG. 3F as a scatter plot with box plot overlay.

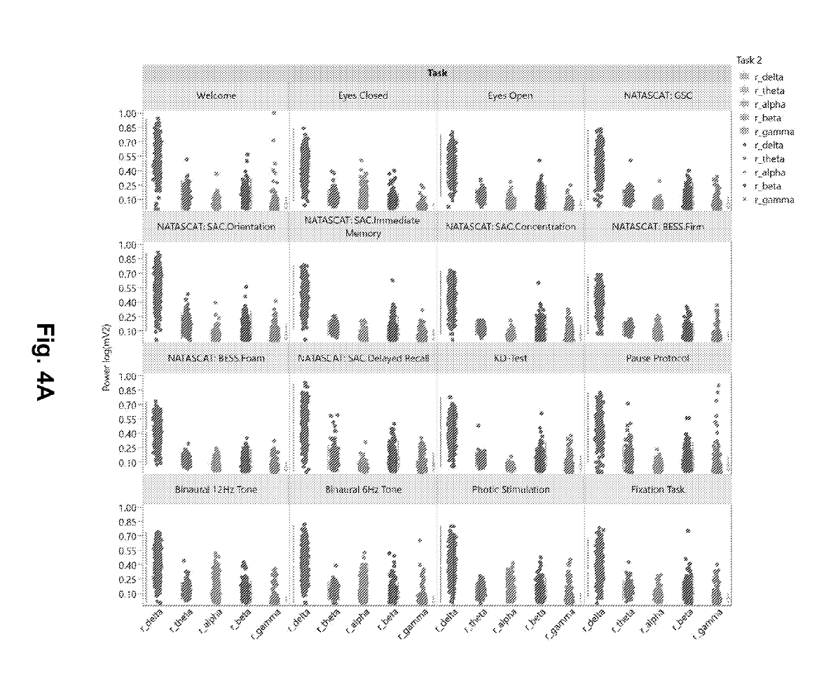

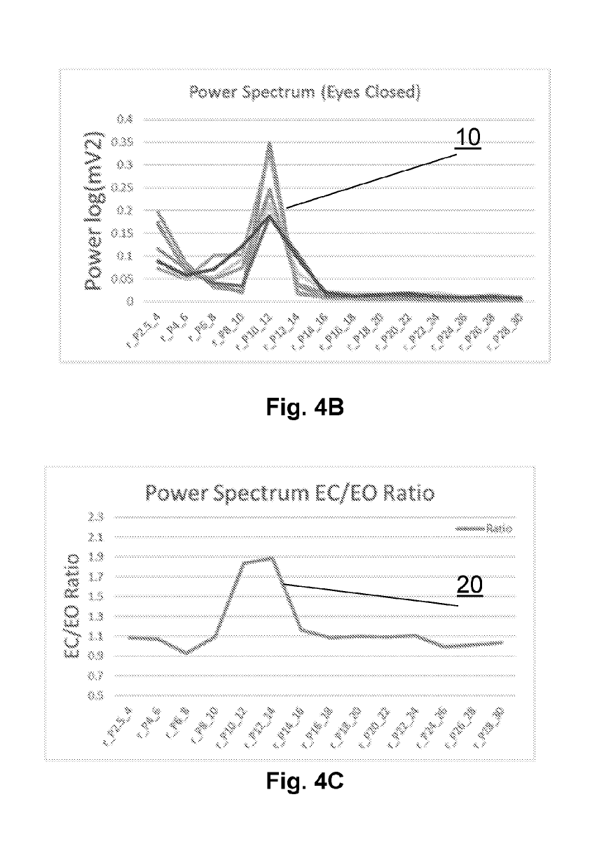

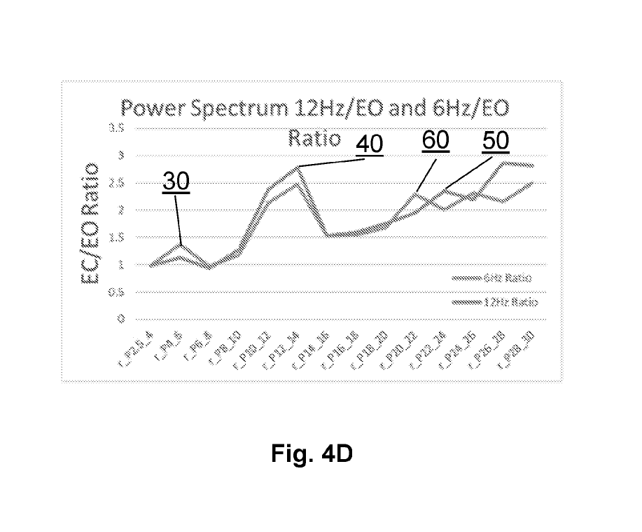

FIG. 4A shows the standard relative spectral EEG band energy in the delta, theta, alpha, beta and gamma bands for each of the 16 tasks in the clinical protocol for the ensemble of baseline scans. In FIG. 4B, one can see the literature expected eyes-closed state alpha band energy elevation 10 in N=7 subjects shown individually. In FIG. 4C, the ratio of the Eyes Closed (EC) spectral band to the Eyes Open (EO) spectral band was calculated for each individual and then averaged over all baseline subjects showing a well-established peak 20. In FIG. 4D, the power spectrum shows nice 6 Hz enhanced power in peak 30 while the other trace shows enhanced energy at 12 Hz in peak 40 as expected by the auditory stimulation of the brain with binaural beats at either 6 Hz (396/403 Hz) or 12 Hz (393/406 Hz).

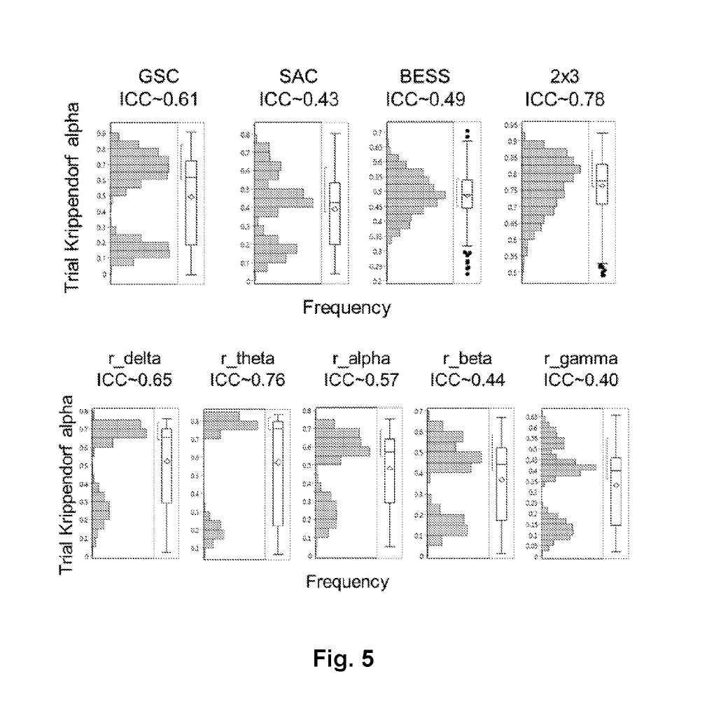

In FIG. 5, the reliability of the individual measures were assessed using a generalization of Pearson's intraclass correlation coefficient developed by Krippendorff as the Krippendorff alpha process. Since there were varying amounts of measurements over the 36 control subjects that were scanned from 6 to 10 times over the episode of care, this more generalized approach was used in an implementation in R-language embedded in JMP Pro with external calls to R. See the following references for details. Krippendorff K (2004). Content Analysis, an Introduction to Its Methodology, 2nd Edition. Thousand Oaks, Calif.: Sage Publications—especially Chapter 11, pages 211-256; Krippendorff K (2004). Human Comm. Res. 30(3): 411-433; Hayes A F and Krippendorff K (2007), Comm. Methods and Measures 1: 77-89.

The data was bootstrapped with 1000 iterations for each measure. The distribution of alphas is shown with the median value cited above each distribution as the ICC approximation or estimate. The four published tasks, GSC, SAC, BESS, 2×3 Saccade are shown in the top row and the 5 primary relative bands of EEG energy are shown in the bottom row. Frequency or count is along the x-axis and the individual Krippendorff alphas calculated are along the y-axis of each task's distribution.

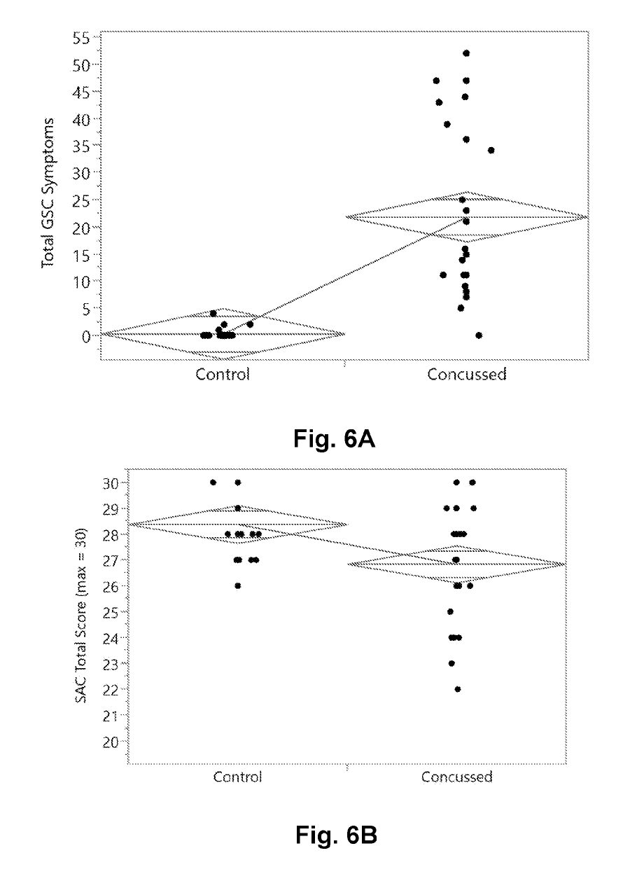

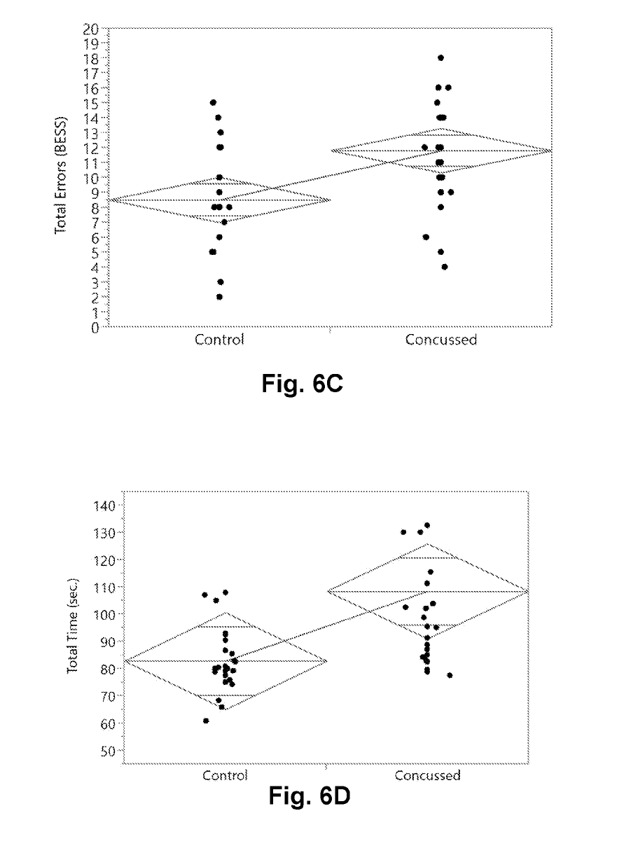

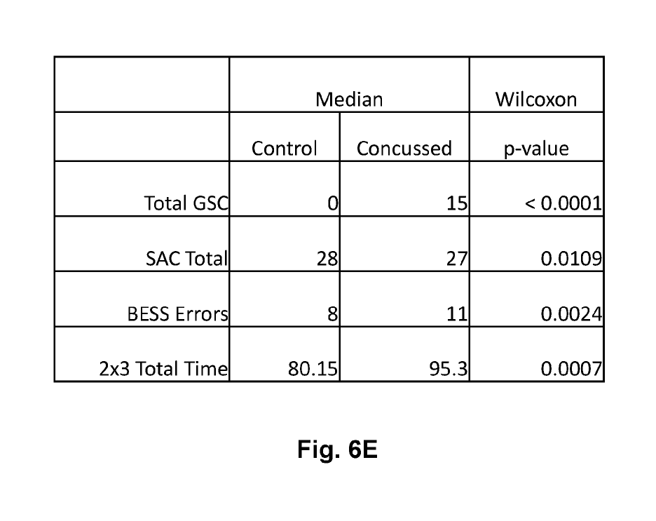

FIG. 6 shows the 2 group comparison of the 4 published tasks with FIG. 6A showing the GSC two group comparison, FIG. 6B showing the SAC, FIG. 6C showing the BESS, and FIG. 6D showing the 2×3 Saccade. FIG. 6E shows a table from the statistical analysis with a Wilcoxon Rank-sum test (non-parametric) test of statistical significance, reporting the false positive rate (FPR) p-value in the far right column.

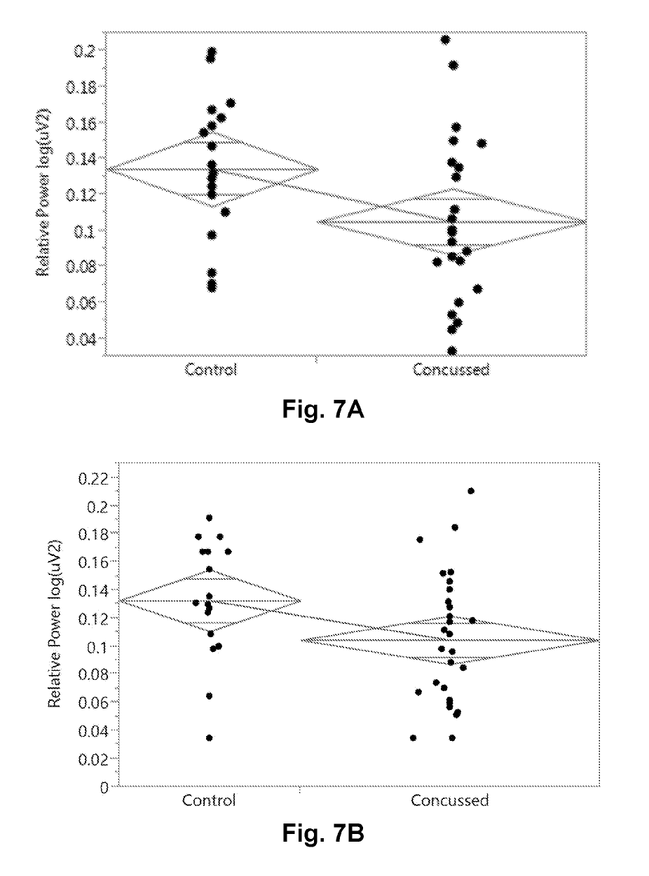

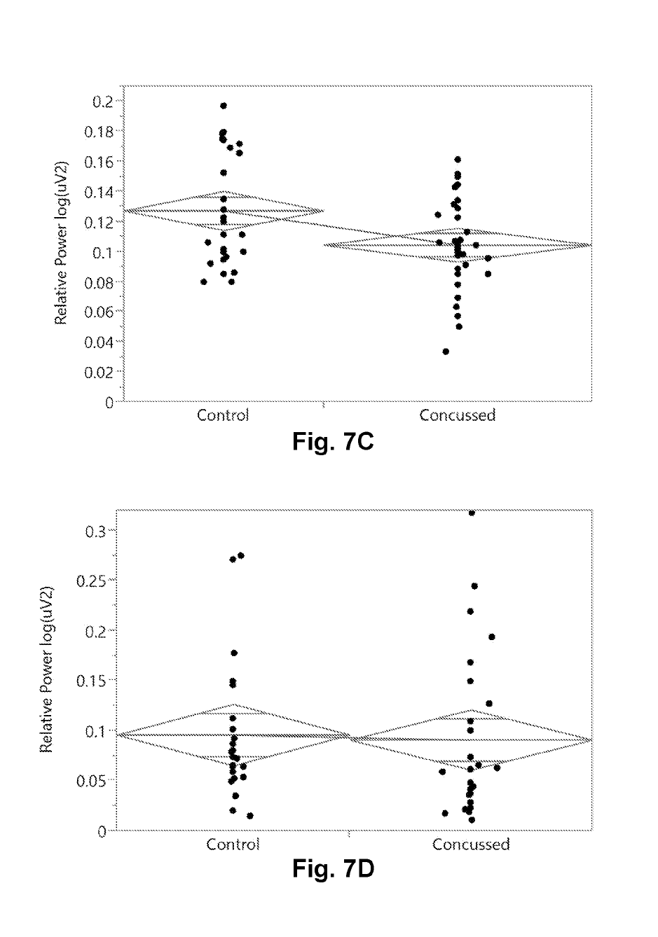

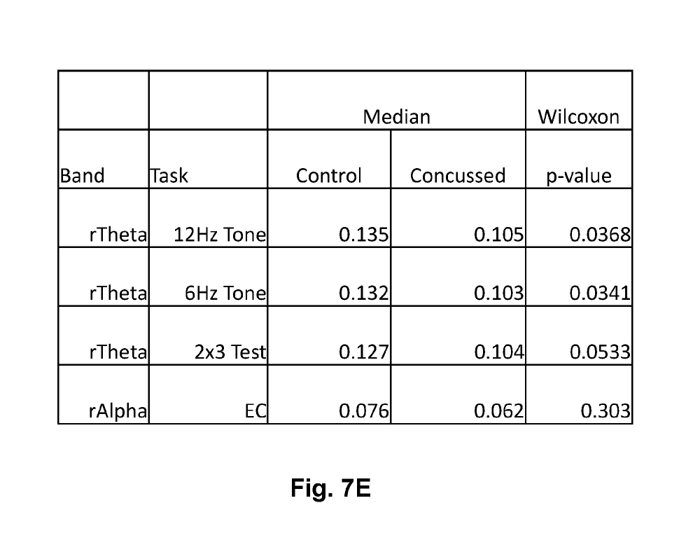

FIG. 7 shows the 2 group comparison of some of the EEG related features that were interesting. In particular, FIG. 7A shows the relative Theta energy down in concussed subjects during the 12 Hz binaural beat task two group comparison. Similarly, FIG. 7B shows the relative Theta energy down in concussed subjects during the 6 Hz binaural beat task two group comparison as well. FIG. 7C shows the relative Theta energy down in concussed subjects during the 2×3 saccade task in the two group comparison. As a negative example, FIG. 7D shows no change in the relative Theta band energy between concussed or control subjects during the Eyes Closed (EC) task. FIG. 7E shows a table from the statistical analysis with a Wilcoxon Rank-sum test (non-parametric) test of statistical significance, reporting the false positive rate (FPR) p-value in the far right column.

In addition, Table 1 below shows additional task-variable or task-feature combinations which in a univariate analysis were statistically different between the concussed and control subjects at the first clinical presentation in the local sports medicine department. In particular, one can see that the relative power in the 38-40 Hz range during the 12 Hz binaural beat task was statistically different, as well as the relative 30-45 Hz power in the same task. As shown in FIG. 7A, the relative Theta power was significantly down in concussed subjects relative to control subjects in the 12 Hz binaural beat stimulation task. Additionally, the relative 4-6 Hz power (or relative Theta-lower) energy was different in the eyes-closed task. During the delayed recall task, it appeared that a lot of artifact was seen observed as different between the concussed subjects and the non-injured comparator subjects at first clinical presentation. The relative 54-56 Hz power in the SAC-concentration task was also different. All these are possible features to be used in predictive models to classify subjects into categories like “injured” or “non-injured” or as features in predictive regression to a concussion index or score. Once the subjects are categorized as non-injured or injured (and the particular injury), medical treatment may be provided to the subject in accordance with the predictive outcome assessment.

Table 1 provides an evaluation of features and tasks looking at the first clinical presentation of the subject to the field clinicians (Scan Visit 1 only), looking at N=94 total subjects, including A=46 TBI and B=48 CTL subjects. All those with statistical probability are shown with the two group ANOVA False positive rate p-value shown in the far right column (Prob>F).

TABLE 1

| Task | Variable | Sum of Squares | Mean Square | F Ratio | Prob > F |
|---|---|---|---|---|---|
| Binaural 12 Hz | r_P38_40 | 0.001 | 0 | 4.8153 | 0.0308 |
| Tone | | | | | |
| Binaural 12 Hz | r_P30_45 | 0.025 | 0 | 4.7195 | 0.0324 |
| Tone | | | | | |
| Binaural 12 Hz | r_theta | 0.008 | 0 | 3.9909 | 0.0487 |
| Tone | | | | | |
| Eyes Closed | r_P4_6 | 0.006 | 0 | 4.6737 | 0.0332 |
| NATASCAT: SAC. | % Artifact | 2876.547 | 2876.5 | 4.5345 | 0.0376 |
| Delayed Recall | | | | | |
| NATASCAT: SAC. | r_P54_56 | 0 | 0 | 4.3912 | 0.0389 |
| Concentration | | | | | |
| NATASCAT: | a_P46_48 | 54291616.98 | 54291617 | 4.2639 | 0.0417 |
| BESS.Firm | | | | | |
| NATASCAT: | a_P48_50 | 18160712.5 | 18160712.5 | 4.1947 | 0.0434 |
| BESS.Firm | | | | | |

These extracted features can then be incorporated into summary feature tables of the present disclosure and used to construct multivariate signatures and classifiers along the with extracted brainwave features, speech recognition features, neuropsychological test data, accelerometer based balance measures, etc.

### EXAMPLES

While the above description contains many specifics, these specifics should not be construed as limitations on the scope of the invention, but merely as exemplifications of the disclosed embodiments. Those skilled in the art will envision many other possible variations that are within the scope of the invention. The following examples will be helpful to enable one skilled in the art to make, use, and practice the invention.

### Example 1. Lehigh University Sports Medicine Concussion Study

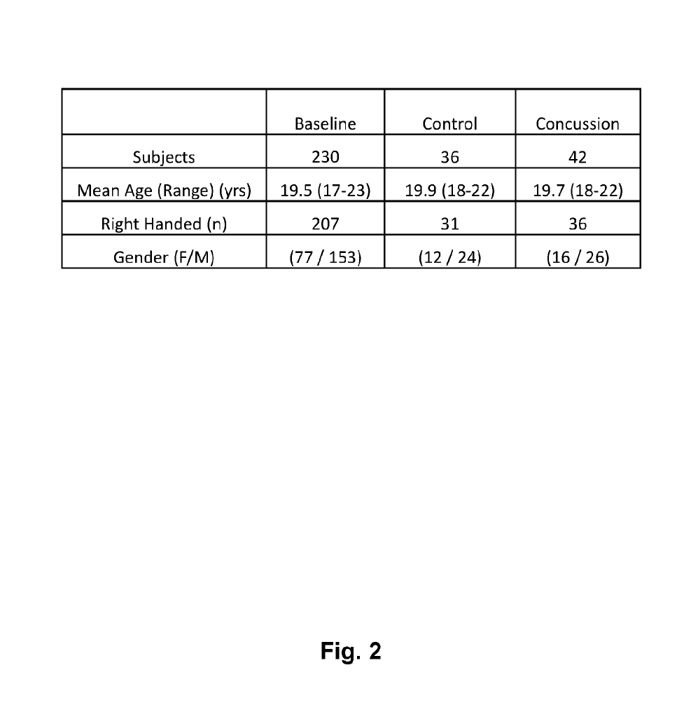

In collaboration with an NCAA Division 1 university, several groups of subjects were enrolled in an Institutional Review Board approved clinical protocol, wherein the first group of subjects (Group A) were clinically diagnosed with a concussion (mTBI) or mild traumatic brain injury, a second control cohort of subjects (Group B) were enrolled who did not have any issue with concussion and served as non-injured Control subjects (CTL), while other athletes from other sports (Group C, etc.) were recruited under the supervision of an Institutional Review Board as well. Group B subjects were recruited within 24 hours of each Group A subject and asked to go through the same scan sequence in time as determined by their brain injured teammate. Participants from groups A, B, C and others were scanned identically with an electronic REM module including a single electrode EEG device as described in U.S. patent application Ser. No. 14/233,292, filed Aug. 6, 2014. The 22-24 minute scan protocol included 1 minute of Eyes Closed, 1 minute of Eyes Open, an automated application of the Graded Symptom Checklist from the SCAT-2, elements of the Standard Assessment of Concussion (SAC) including orientation, immediate memory recall, concentration, delay memory recall, a full Balance Error Scoring System (on both firm and foam surfaces), King-Devick Test Cards, binaural beat audio stimulation at 6 and 12 hertz beat frequency centered at 400 Hz, photic stimulation, and a fixation task including a moving red cross for 1 minute. For the purposes of an initial analysis, FIG. 2 reports the demographics in the initial analysis of the 3 year study data.

The stop watch times and errors for each card of the King-Devick test were recorded manually by the test administrators while the peripheral MCU (a Dell Vostro 3550 laptop computer) presented the cards and recorded the responses of the individuals via the microphone and mouse clicks. The BESS errors were recorded manually as well as the SAC responses. The head based REM module continuously recorded the forehead EEG from 10-20 montage position Fp1 relative to mastoid on the left ear for reference REF and ground GND. A multi-modal assessment consisting of an EEG data stream, a cognitive data stream (reaction time and accuracy), self-report of concussion symptoms, and a microphone data stream were recorded depending upon which tasks were being conducted. The data was encrypted locally before being transported over a secure connection data pipe to a secure virtual server in cyberspace.

Signal analysis scientists were blinded to participant clinical diagnosis for the purposes of artifact detection, signal processing and feature extraction. The extracted feature data table was then quality controlled and scrubbed to remove as many errors as possible. The total time for the King-Devick test was calculated according to the published procedure of using the minimal number of errors and then summing the individual times to read all three cards in succession. This total time represents one extracted variable and underwent a logistic classification model. Serial assessments were conducted on both concussed athletes and controls with from three to up to ten scans assessing both concussed and controls.

### Example 2. Artifact Detection Pre-Processing and Signal Processing of the EEG Data

EEG data was loaded into memory within MATLAB (Mathworks, Natick, Mass.) for preprocessing and signal processing activities.

Preprocessing occurred to remove samples that contained artifacts. The EEG data can be viewed as an alternating current signal. The EEG data was bandpass filtered with a least squares Finite Input Response filter with Stopband Frequencies of 0.5 Hz and 42.0 Hz and Passband Frequencies of 1.0 Hz and 45.0 Hz. Stopband and Passband weights were set to 1.0. The filter was applied twice to achieve a 2-fold attenuation and 0-phase shift, first in the temporal direction of signal collection and again in the reverse order of the collected data. The mean (X-bar) and standard deviation (STD) of the filtered signal is calculated for all data collected in a recording session. The value of the signals STD was multiplied by a constant value set by the user or was built into the settings for the algorithm. All signal values samples that exceed the multiplied STD value (both positive values and negative values) were marked as Artifact. All adjacent signal value samples that were identical and exceed a predetermined length (number of samples by the user) were marked as Artifact as well. All identified types of artifact were combined into a single Artifact type. Artifacts that occur in time within a user identified limit were combined as a single duration of artifact that included the beginning of the 1^{st} Artifact and the end of the 2^{nd} Artifact. Signal data was also marked as Artifact between the end of the identified Artifact and the point at which the signal crosses the value “0”. Additionally, Signal data was also marked as Artifact between the beginning of the identified Artifact and the prior point at which the signal crosses the value “0”. Spectral components of the Signal local to the Artifact were estimated with a fast Fourier transform (FFT). Original Signal data samples marked as artifact were replaced with a synthetic signal generated from the calculated spectral components.

The artifacts were removed from the recorded signal in preprocessing. Next, after denoising the signal, the power spectral density was calculated separately for each data segment or block in a recording, typically between 30 seconds and 3 or 4 minutes per block of data per task. The power spectrum was calculated by segmenting the data (range 5-15 seconds, typically 10 seconds), applying an antialias filter with a bandpass from 1.0 Hz to the Nyquist frequency (typically 256 Hz since data was typically gathered at 512 samples per second), convolving the data segment with a Blackman window function, and applying the FFT algorithm. Data segments consisted of windows of data that overlap by 95% with a sliding or rolling process down a full block of data. The geometric mean of the power spectrums from all overlapping sliding window data segments was calculated to generate a single absolute power spectrum for any given block of recorded data. The absolute spectral power values were used to generate an additional set of signal features. First the absolute spectral power values were divided by the total spectral power to calculate the relative power spectrum.

Next the absolute and relative power spectra were used to extract biomarker features for analysis. In the feature extraction process, first the absolute and relative spectral power values were summated within the following well established ranges to form typical energy bands as reported in the literature: (delta 1-4 Hz; theta 4-8 Hz; alpha 8-12 Hz; beta 12-30 Hz; gamma>30 Hz). Second, ratios of the absolute or relative summed power in these bands were calculated to produce additional candidate features including: theta/alpha, delta/alpha, theta/beta, delta/beta, theta/(alpha+beta), delta/(alpha+beta), (delta+theta)/(alpha+beta). Third, absolute or relative spectral power was summed in small frequency bins including 2.5-4 Hz and in 2 Hz bins from 4 Hz to 60 Hz as alternate features. Finally, the power spectrum mean, STD, skewness, and kurtosis were calculated as features.

Similar processing was done using Discrete Wavelet Transforms and Continuous Wavelet transforms as published and additional features extracted. All features were exported to a .csv or .txt file for import into well-established statistical analysis software packages. JMP Pro from SAS (Cary, N.C.) was used most commonly but the R-language and Matlab statistical software was used as well.

### Example 3. Baseline Characterization

As described earlier, FIG. 3 presents the data observed for the published concussion instruments built into the clinical study. The GSC, SAC, BESS and 2×3 Saccade results show broad variation depending on the type of scale. In some instances, there are floor (2×3 Saccade errors) as well as ceiling (SAC) effects observed.

FIG. 4 shows the baseline characterization of the EEG data in the five primary bands in each of the 16 tasks of the clinical protocol.

### Example 4. Measures of Reliability and Validity of the EEG Data

As described above, FIG. 4B, FIG. 4C and FIG. 4D each provide nice corroboration of an expected observation. In the case of FIG. 4B, one sees prominent alpha peaks 10 in the Eyes Closed spectra, consistent with much published literature. In FIG. 4C, the EC/EO ratio across all baseline subjects shows a nice prominent peak 20. Lastly, when driven with a 6 Hz binaural beat, FIG. 4D shows an elevation peak at 6 Hz 30, while when driven by a 12 Hz binaural beat, FIG. 4D shows an elevation peak at 12 Hz 40. Thus, the driving beat frequency is observed in the baseline subjects and can serve as a candidate feature for inclusion in predictive models of classification or regression. Interestingly, it should be noted that the first harmonic of 6 Hz binaural beat stimulation was also observed as there is a peak 40 in the 6 Hz Binaural Beat trace as well as the one observed in the 12 Hz trace in FIG. 4D. One can further note a slight elevation at the first harmonic of the 12 Hz binaural beat stimulation observed as a peak 50 at 24 Hz as well as a slight elevation 60 at the 2^{nd} harmonic of the 6 Hz binaural beat stimulation near 18 Hz. Together, these data provide evidence of the validity of the EEG measures. In particular they provide support for the use of a primary driving frequency or its first harmonic in a binaural beat stimulation task.

FIG. 5 shows reliability estimates using a generalization of Pearson's Intraclass Correlation coefficient (or ICC) using the Krippendorff alpha formalism as earlier described. One sees comparable reliability between the 5 primary EEG bands as for the four well published literature instruments or tools (GSC, SAC, BESS, 2×3 Saccade).

### Example 5. Identification of Significantly Different Features Between Brain Injury and Non-Injured Subjects

FIG. 6 validates the literature reported tools ability to distinguish on average the concussed versus control subjects. All four tools appear to meet statistical significance (Wilcoxon rank-sum non-parametric method). In addition, FIG. 7 and Table 1 above identify statistically significant features to be used in predictive models to classify subjects into categories or conduct regression to a numeric index. All these features and the tasks that they are associated with can be utilized alone or in multivariate combination with the published features of FIG. 6 to create multimodal multivariate predictive models.

### Example 6. Creation of Predictive Regression and Classification Models (Prophetic)

Creation of predictive models to both classify subjects based on their extracted features from an individual scan session of the 16 task battery described in FIG. 1 can be done. Features would be extracted according to the previously described data collection and analysis. Individual features, alone or in multivariate combination would be used in predictive methods known in the field such as logistic regression, tree based methods such as random forest, boosted/bagged trees, decision trees, discriminant analysis such as linear or quadratic discriminant analysis, or support vector machines, machine learning, or neural nets. Standard techniques such as K-fold internal cross-validation can be employed before external validation data sets are available. Further work is necessary on these data.

### Example 7. Generation of Clinical Report

Once the extracted features have been determined, the extracted features can be put first into a clinical report. In addition, the extracted features can be put into a classification or regression predictive model to provide additional information and insight to the licensed health care professional. This would further include searching of previous cases and reporting of successful therapies learned previous cases and information. This would include the standard machine learning approaches, such as support vector machines, neural networks, genetic algorithms, logistic regression, and tree-based predictive models (e.g. random forest).

Those skilled in the art will also appreciate that the present disclosure may be applied to other applications and may be modified without departing from the scope of the present disclosure. For example, the signal processing described herein may be performed on a server, in the cloud, in the electronics module, or on a local PC, tablet PC, smartphone, or custom hand held device Accordingly, the scope of the present disclosure is not intended to be limited to the exemplary embodiments described above, but only by the appended claims.

1. A method of assessing brain function, comprising:
electronically recording biologic information of a user with one or more electronics modules as the user progresses through a series of cognitive, sensory, activation, and/or stimulation tasks;
extracting one or more data features from the record biologic information to obtain extracted data features;
analyzing the extracted data features for each task so to develop a predictive outcome assessment of one or more brain conditions of the user, wherein predictive outcome assessment is at least one of a) an injury determination, b) a brain injury index, or c) a brain health assessment; and
providing medical therapy to the user in accordance with the predictive outcome assessment.

2. The method of claim 1, wherein the injury determination is either categorization of an injury or a non-injury.

3. The method of claim 1, wherein the brain injury index is a concussion index.

4. The method of claim 1, wherein the series of stimulation tasks in part comprise a Lehigh protocol.

5. The method of claim 1, wherein one extracted data feature or multiple extracted data features are used to a compile the predictive outcome assessment.

6. The method of claim 1, wherein the biologic information is an EEG data stream, a cognitive data stream of reaction time and accuracy, a self-report of concussion symptoms, a microphone data stream, and an accelerometer based balance data stream.

7. A system comprising:
one or more electronics modules configured to be mounted on the user, the one or more electronics modules including an active brainwave sensor that collects at least one channel of an electroencephalography (EEG) brainwave data stream;
a plurality of biological sensors that simultaneously record biological sensor data from the user, said plurality of biological sensors including a microphone that records human speech to capture verbal responses of the human subject during a series of tasks, and an image sensor that records eye movements, eye saccade and biometric identification information; and
a stimulation device that applies at least one of a visual stimulant, an auditory stimulant, a gastronomic stimulant, an olfactory stimulant, and/or a motion stimulant to the user,
wherein the plurality of biological sensors simultaneously measure the user's response to stimulants applied by said stimulation device in accordance with at least one task that causes statistically different results between brain injured subjects and brain non-injured subjects for recordation by said electronics module.

8. The system of claim 7, wherein the at least one task comprises a binaural 12 Hz beat task and said electronics module measures at least one of relative power in a 38-40 Hz range during a binaural 12 Hz beat task, relative power in a 30-45 Hz range during a binaural 12 Hz beat task, and a relative theta power during a binaural 12 Hz beat task.

9. The system of claim 7, wherein the at least one task comprises an eyes closed task and said electronics module measures relative 4-6 Hz power or relative theta-lower power during the eyes closed task.

10. The system of claim 7, wherein the at least one task comprises a Standardized Assessment of Concussion (SAC)-delayed recall task and said electronics module measures artifact during the SAC-delayed recall task.

11. The system of claim 7, wherein the at least one task comprises a Standardized Assessment of Concussion (SAC)-concentration task and said electronics module measures relative 54-56 Hz power during the SAC-concentration task.

12. The system of claim 7, wherein the at least one task comprises a Balance Error Scoring System (BESS) firm surface task and said electronics module measures at least one of absolute 46-48 Hz power during the BESS firm surface task and absolute 48-50 Hz power during the BESS firm surface task.

13. The system of claim 7, wherein the at least one task comprises a binaural 6 Hz beat task and said electronics module measures a 6 Hz binaural beat primary driving frequency and a first harmonic.

14. The system of claim 7, wherein the at least one task comprises a binaural 12 Hz beat task and said electronics module measures a 12 Hz binaural beat primary driving frequency and a first harmonic and/or a second harmonic.
